(19) United States
(12) Plant Patent Application Publication
(10) Pub. No.: US 2019/0014706 P1
(43) Pub. Date: Jan. 10, 2019

(54) Lupinus 'LSDOR04-0'

(50) ;

(71) Applicant: Kazunori Sato, Tokyo (JP)

(72) Inventor: Kazunori Sato, Tokyo (JP)

(21) Appl. No.: 15/998,135

(22) Filed: Jul. 5, 2018

Related U.S. Application Data
(60) Provisional application No. 62/604,473, filed on Jul. 8, 2017.

(51) Int. Cl. A01H 6/54

(52) U.S. Cl. PLT/263.1

(57) ABSTRACT

A new and distinct Lupinus cultivar named ‘LSDOR04-0’ is disclosed, characterized by distinctive salmon and pink flower coloration. Plants uniquely bloom under both short day and long day conditions without vernalization. The new cultivar is a Lupinus, suitable for ornamental purposes.

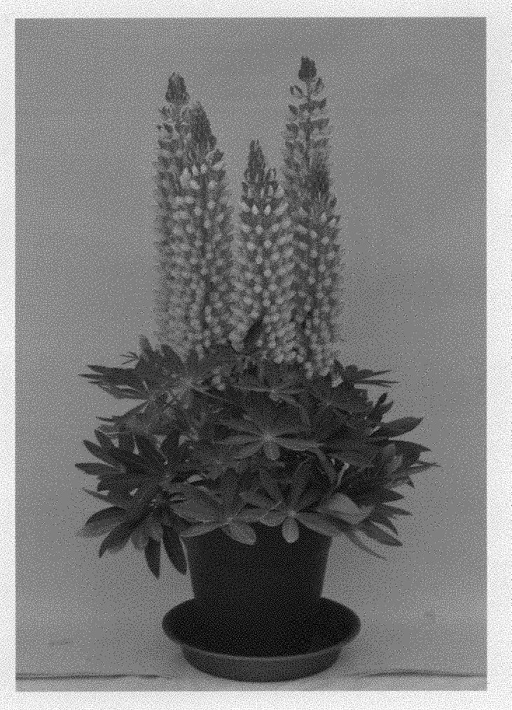

Latin name of the genus and species: Lupinus hybrida.

Variety denomination: ‘LSDOR04-0’.

### BACKGROUND OF THE INVENTION

new variety, hereinafter referred to as ‘LSDOR04-0’, was discovered as part of a planned breeding program. The intent of the breeding program was to develop Lupinus hybrids for commercial ornamental applications.

### SUMMARY OF THE INVENTION

The cultivar ‘LSDOR04-0’ has not been observed under all possible environmental conditions. The phenotype may vary somewhat with variations in environment such as temperature, day length, and light intensity, without, however, any variance in genotype. The following traits have been repeatedly observed and are determined to be the unique characteristics of ‘LSDOR04-0’ These characteristics in combination distinguish ‘LSDOR04-0’ as a new and distinct Lupinus cultivar:
1. Plants uniquely bloom under both short day and long day conditions without vernalization.
2. Unique salmon and pink flower coloration.

### COMPARISON TO PARENT VARIETY

‘LSDOR04-0’ is similar in most horticultural characteristics to the seed parent variety. Plants of the new cultivar ‘LSDOR04-0’ however, differ in the following characteristics:
1. The new variety has a different flower color.

‘LSDOR04-0’ is similar in most horticultural characteristics to the pollen parent variety. Plants of the new cultivar ‘LSDOR04-0’ however, differ in the following characteristics:
1. The new variety has a different flower color.

### COMMERCIAL COMPARISON

‘LSDOR04-0’ can be compared to Lupinus citrunus unpatented. Plants of the new cultivar ‘LSDOR04-0’ are similar to plants of this comparator in some characteristics, however, the new variety differs in the following:
1. Flowers of the new variety have more pink tones, this comparator has yellow or white and yellow flowers.
2. Plants of the new variety are taller.
3. Plants of the new variety flower more abundantly, both with longer and more inflorescences.

‘LSDOR04-0’ can be compared to the commercial variety Lupinus ‘West Country Salmon Star’ unpatented. Plants of the new cultivar ‘LSDOR04-0’ are similar to plants of ‘West Country Salmon Star’ in most horticultural characteristics, a Plants of the new cultivar ‘LSDOR04-0’ however, differ in the following:
1. Flower color of the new variety has more pink tones.
2. Plants of the new variety are shorter and sturdier.
3. Plants of the new variety will flower without vernalization, as required by this comparator.

### BRIEF DESCRIPTION OF THE PHOTOGRAPH

The accompanying photograph in illustrates in full color a typical plant of ‘LSDOR04-0’ grown in a greenhouse in Santa Paula, Calif. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007 except where general terms of ordinary dictionary significance are used. The following observations and measurements describe ‘LSDOR04-0’ plants grown outdoors in Santa Paula, Calif. Plants are approximately 1 year old, in a 2-gallon nursery container. Measurements and numerical values represent averages of typical plant types.
Botanical classification: Lupinus hybrida ‘LSDOR04-0’.
Propagation: Typically by shoot tissue culture.
   Root description.—Single main root, moderate branching of short lateral roots. Cream to tan in color, not accurately measured with an R.H.S. chart.
Plant:
   Growth habit.—Compact and herbaceous vigorous perennial. Mounding foliar base topped with upright raceme inflorescences.
   Height.—Approximately 25 cm to top of foliar plane, approximately 60 cm to top of flowering plane.
   Plant spread.—Approximately 35 cm.
   Branching characteristics.—No true branching. Leaves emerge direct from base of plant, or infrequently occur directly on the flowering stem.
   Age of plant described.—Approximately 1 year.
Foliage:
   Leaf.—Palmately compound. Occurring mainly as basal whorls. Infrequently alternate along flowering stem. Shape: Orbicular. Length: Average range 10.0 to 12.0 cm. Diameter: Average range 10.0 to 15.0 cm. Quantity: Approximately 20 to 25 basal leaves, per plant. Average range 4 to 8 floral stem leaves, per stem.
   Leaflets.—Quantity: Average 11. Shape: Oblanceolate. Arrangement: Radial. Average Length: Longest leaflet per leaf average 6.0 to 8.0 cm. Shortest leaflet per leaf average 4.5 cm. Average Width: Central leaflet average 1.9 to 2.3 cm. Apex: Acuminate. Base: Attenuate. Margin: Entire. Texture of top surface: Glabrous. Texture of bottom surface: Glabrous. Appearance of top surface: Matte. Appearance of bottom surface: Matte. Aspect: Flat to very slightly reflexed downward. Color: Upper side: Near RHS Green 137A. Under side: Near RHS Green 138A. Venation: Type: Pinnate. Venation color upper side: Near RHS Yellow-Green 145A. Venation color under side: Near RHS Yellow-Green 145B. Petiole: Length: Average range 6.0 to 16.0 cm. Diameter: 0.5 cm. Color: Near RHS Yellow-Green 145B. Texture: Minutely hirsute.
Flower:
   Natural flowering season.—Spring until Summer.
   Inflorescence type and habit.—Single large papilonaceous flowers evenly and symmetrically arranged on racemes.
   Flower longevity on plant.—Individual flowers approximately 15 days on the plant, not persistent.
   Quantity of flowers.—60 to 110 flowers and buds per inflorescence. Typically 4 to 6 inflorescences per plant at one time.
   Inflorescence size.—Diameter: Approximately 7.5 cm. Height: Average range 25 to 45 cm.
   Flower size (excluding pedicel).—Length: Approximately 1.9 cm. Height: Average range 1.7 cm.
   Peduncle.—Length: Approximately 7.0 cm from uppermost leaf to lowermost flower. Diameter: Approximately 0.8 cm. Color: Near RHS Yellow-Green N144D. Strength: Flexible, very strong. Texture: Short pubescence.
   Pedicel.—Length: Approximately 1.0 cm. Diameter: Approximately 0.2 cm. Color: Near RHS Yellow-Green 145B. Strength: Strong. Texture: Softly pubescent.
   Petals.—Quantity and arrangement: 1 upper banner, 2 lateral “wings” and a lower keel. Banner: Length: Approximately 1.7 cm. Width (furled, natural state): Approximately 0.8 cm. Shape: Orbicular, when unfurled. Margin: Entire. Apex: Mucronate. Base: Obtuse. Texture: Smooth, glabrous all surfaces. Aspect: Folded, margins furled. Banner Color: When opening: Outer surface: Near RHS Yellow 4A. Inner surface: Near RHS Yellow 4C. Fully opened: Outer surface: Near RHS Yellow 8B, center stripe 7A. Inner surface: Near RHS Yellow 9D, center stripe 8A. Fading: Outer surface: Near RHS Yellow 8B, center stripe 8A. Inner surface: Near RHS Yellow 9D, center stripe 8A. Wing: Length: Approximately 1.5 cm. Width): Approximately 1.0 cm. Shape: Ovate. Margin: Entire. Apex: Rounded, frequently fused to opposite wing at apex. Base: Truncate. Texture: Smooth, glabrous all surfaces. Aspect: Inwardly cupped. Wing Color: When opening: Outer surface: Near RHS Yellow 12C, flushed Orange-Red N34C. Inner surface: Near RHS Yellow 12D, slightly flushed Orange-Red N34C. Fully opened: Outer surface: Near RHS Orange-Red 35A, stripes yellow 12C. Inner surface: Near RHS Yellow 13D, stripes Orange-Red 35C. Fading: Outer surface: Near RHS Orange-Red 35B, stripes yellow 12C. Inner surface: Near RHS Yellow 13D, stripes Orange-Red 35C. Keel: Length: Approximately 1.2 cm. Width): Approximately 0.5 cm. Shape: Scythe. Margin: Entire. Apex: Awn-like. Base: Truncate. Texture: Smooth, glabrous all surfaces. Aspect: Folded. Keel Color: When opening: Outer surface: Near RHS Yellow 11D, apex Greyed-Purple 187A. Inner surface: Near RHS Yellow 11D, apex Greyed-Purple 187A. Fully opened: Outer surface: Near RHS Yellow 4D, apex Greyed-Purple 187A. Inner surface: Near RHS Yellow 4D, apex Greyed-Purple 187A. Fading: Outer surface: Near RHS White 155D, Greyed-Purple 187A. Inner surface: Near RHS White 155D, Greyed-Purple 187A.
   Bud.—Shape: Flattened ovate. Length: Approximately 1.1 cm. Diameter: Approximately 0.9 cm. Color: Near RHS Green-Yellow 1C. Sepals: Quantity: 2, one fully developed, one minute (less than 1 mm in length), sometimes missing. Length: Approximately 6 mm. Width: Approximately 4 mm. Shape: Deltate. Aspect: Cupped. Apex: Mucronate. Margin: Entire. Color. Interior Surface: RHS Yellow-Green 145C. Exterior Surface: RHS Yellow-Green 145C. Texture: Glabrous. Fragrance: Strong, sweet scent.
Reproductive organs:
   Stamens.—Number: Approximately 8. Filament length: Approximately 0.5 cm. Filament color: Near RHS Yellow-Green 145D.
   Anthers.—Length: 0.2 cm. Shape: Elongated ovoid. Color: Near RHS Yellow-Green 145B. Pollen: Scant, colored near Yellow-Orange 21A.
   Pistil.—Number: 1. Length: Approximately 1.1 cm. Stigma: Shape; Globular. Color: Near RHS Yellow-Green 145D. Ovary: Near RHS Yellow-Green 145C, approximately 2 mm in diameter.
Other characteristics:
   Disease and pest resistance.—Not observed to be susceptible nor resistant to normal diseases and pests of Lupinus.
   Temperature tolerance.—Typically USDA Zones 3 to 9.
   Fruit/seed production.—Not observed.

What is claimed is:

1. A new and distinct cultivar of Lupinus plant named ‘LSDOR04-0’ as herein illustrated and described.
